(19) United States
(12) Patent Application Publication
(10) Pub. No.: US 2016/0324431 A1
(43) Pub. Date: Nov. 10, 2016

(54) Flexible Optic Fiber Sensor Film, Mat Structure Comprising the same and Method of Use of the Mat Structure

(71) Applicant: Ospicon Company Limited, Hong Kong (HK)

(72) Inventors: Seng Tat NG, Singapore (SG); Hwee Siong CHONG, Singapore (SG); Xin JIANG, Singapore (SG); Xu LIN, Shenzhen (CN)

(73) Assignee: Ospicon Company Limited, Hong Kong (HK)

(21) Appl. No.: 14/906,241

(22) PCT Filed: Dec. 4, 2014
(86) PCT No.: PCT/CN2014/093043

§ 371 Date: Jan. 19, 2016

(51) Int. Cl. A61B 5/024; G01D 5/353; A61B 5/11; A61B 5/00; A61B 5/08

(52) U.S. Cl. CPC A61B 5/02416; A61B 5/6892; A61B 5/0816; A61B 5/11; G01D 5/353; A61B 5/0062; A61B 2560/0214

(57) ABSTRACT

A flexible optic fiber sensor film, a mat structure comprising the same and a method of use of the mat structure are provided. The flexible optic fiber sensor film comprises a sandwiched layer and an optic fiber cable arranged in the sandwiched layer; the flexible optic fiber sensor film further comprises protrusions arranged on the sandwiched layer to abut against the optic fiber cable. The flexible optic fiber sensor film is configured for generating light loss in the optic fiber cable when there are body movements of a human subject lying on top of the flexible optic fiber sensor film. The application is safe and comfortable to the human subject.

### FIELD OF THE INVENTION

The present invention relates to a flexible optic fiber sensor film for detection of one or more of vital signs of human subjects, and to a mat structure comprising the flexible optic fiber sensor film and a method of use of the mat structure.

### BACKGROUND OF THE INVENTION

Currently, there are piezoelectric sensors that are used for measurement of respiration rate, heart rate and movement of human subjects sleeping on a bed/mattress. Normally, the piezoelectric sensor is in the form of a sensor pad inserted below the bed/mattress. The piezoelectric sensor has a very high DC output impedance and can be modeled as a proportional voltage source and a filter network. As shown in FIG. 1, a voltage output is directly proportional to an applied force, pressure or strain. Depending on the type of piezoelectric material used, the output voltage range versus the strain/pressure may vary. Piezoelectric sensors can be made of piezoelectric ceramics (PZT ceramics) or single crystal materials. These materials are hard and have a sensitivity that degrades over time. This degradation is highly correlated with increasing temperature. These piezoelectric sensors also tend to be sensitive to more than one physical factor and tend to show a false signal when they are exposed to vibrations. Another major disadvantage of piezoelectric sensors is that they cannot be used for truly static measurement. A static force will result in a fixed amount of charges on the piezoelectric material, which means that the output voltage of the piezoelectric sensor disappears once the force/weight has reached a steady state.

### SUMMARY OF THE INVENTION

The objective of the present invention is to provide a flexible optic fiber sensor film for detection of presence, movement, respiration rate and heart rate of human subjects, a mat structure and a method of use of the mat structure, aiming at overcoming the defects that materials of the piezoelectric sensors are hard and have a sensitivity that degrades over time, and the piezoelectric sensors cannot be used for truly static measurement.

The technical solutions of the present invention for solving the technical problems are as follows:

In one aspect, a flexible optic fiber sensor film is provided. The flexible optic fiber sensor film comprises a sandwiched layer and an optic fiber cable arranged in the sandwiched layer. The sandwiched layer comprises an upper film and a lower film; the optic fiber cable is sandwiched between the upper film and the lower film. Protrusions are arranged on the upper film and the lower film to abut against the optic fiber cable and configured for generating light loss in the optic fiber cable when there are body movements of a human subject lying on top of the flexible optic fiber sensor film.

In one embodiment, the protrusions on the upper film and the protrusions on the lower film are face-to-face, to press directly onto the optical fiber cable.

In another embodiment, two pieces of protection films are inserted in the sandwiched layer and sandwich the optic fiber cable.

In another embodiment, both the protrusions on the upper film and the protrusions on the lower film face to one direction, such that only the protrusions on the upper film or only the protrusions on the lower film directly press onto the optic fiber cable.

In another embodiment, one piece of protection film is inserted in the sandwiched layer and is between the optic fiber cable and one of the upper film and the lower film such that the optic fiber cable does not contact the protrusions.

In another embodiment, the upper film and the lower film are back-to-back, such that none of the protrusions comes into contact with the optic fiber cable.

In another aspect, a mat structure comprising the flexible optic fiber sensor film is provided. The mat structure further comprises a programmable LED driver, a light source, a light sensor and a processor. An output terminal of the programmable LED driver is connected to the light source, and the light source is connected to one terminal of the optic fiber cable, and the other terminal of the optic fiber cable is connected to the light sensor; the processor is configured for delivering a control signal to drive the programmable LED driver to supply a LED current to the light source; the light source is configured for generating light by flow of the LED current and piping the light into the optic fiber cable; the light sensor is configured for detecting a light loss signal caused in the optic fiber cable. The processor is also configured for processing the light loss signal derived from the light sensor for detection of vital signs.

In one embodiment, the processor, the programmable LED driver, the light source, and the light sensor are integrated into a head unit electronic assembly. The head unit electronic assembly further accommodates a dry cell battery configured for supplying power to the programmable LED driver, the light sensor and the processor.

In another embodiment, the processor, the programmable LED driver, the light source, and the light sensor are integrated into an electronic box. The flexible optic fiber sensor film is attached to the electronic box via an optical fiber protective sleeve. The electronic box is powered via an AC adapter connected to a wall AC supply.

In another embodiment, the mat structure further comprises a protective layer below the flexible optic fiber sensor film and an outer mat cover that encases the flexible optic fiber sensor film and the protective layer.

In another embodiment, the protective layer comprises multiple strips spaced with defined gaps in between.

The upper film, the lower film, the protection films, the protrusions, the protective layer, and the outer mat cover are made of elastic material selected from plastic, rubber, nylon, particularly polyethylene.

In another aspect, a method of detecting presence of a human body by using the mat structure, comprising a step of detecting a sudden DC signal spike or drop of the light loss signal is provided.

In another aspect, a method of respiration rate measurement by using the mat structure, comprising a step of identifying AC components of the light loss signal with each pulse represented as one breath count in time domain is provided.

In another aspect, a method of heart rate measurement by using the mat structure, comprising a step of extracting a heart rate signal by identifying AC (alternating signal) components of the light loss signal in frequency domain is provided.

When implementing the present invention, the following advantageous effects can be achieved: the flexible optic fiber sensor film of the present invention can generate light loss for detection of presence, movement, respiration rate and heart rate of human subjects via the protrusions, and the present invention uses the protection film to protect the optic fiber cable. The mat structure of the present invention adopts the head unit electronic assembly together with the flexible optic fiber sensor mat as one unit to be used for infant applications, and adopts the electronic box attached to the flexible optic fiber sensor mat via the optical fiber protective sleeve for adult applications. The invention can be configured for detection of presence, movement, respiration rate and heart rate of human subjects, and is safe and comfortable to the human subject.

### BRIEF DESCRIPTION OF THE DRAWINGS

FIG. 1 is an equivalent circuit diagram of a piezoelectric sensor;

FIG. 2 is a schematic diagram of a flexible optic fiber sensor film and its external light source;

FIG. 3 is a schematic diagram of a flexible optic fiber sensor film embedded in a mattress;

FIG. 4 is a schematic diagram of a flexible optic fiber sensor film embedded into a pillow;

FIG. 5 is a schematic diagram of a flexible optic fiber sensor film placed below the pillow;

FIG. 6 is a schematic diagram of a flexible optic fiber sensor mat with a head unit electronic assembly;

FIG. 7 is a schematic diagram of a rolled flexible optic fiber sensor mat shown in FIG. 6;

FIG. 8 shows an application of the flexible optic fiber sensor mat shown in FIG. 6;

FIG. 9 is a schematic diagram of a flexible optic fiber sensor mat with an electronic box;

FIG. 10 is a schematic diagram of a rolled flexible optic fiber sensor mat shown in FIG. 9;

FIG. 11 shows an application of the flexible optic fiber sensor mat shown in FIG. 9;

FIG. 12A is a schematic diagram of a Protective Layer of the present invention;

FIG. 12B is a cross sectional view of a flexible optic fiber sensor mat of the present invention;

FIG. 12C is cross sectional view of a bended flexible optic fiber sensor mat of the present invention;

FIG. 13 is an exploded view of a flexible optic fiber sensor film of the present invention;

FIG. 14 is a perspective view of the sandwiched layer of the present invention;

FIG. 15 is a cross sectional view of a flexible optic fiber sensor film illustrating the protrusions on an upper and a lower film abutted against an optic fiber cable;

FIG. 16 shows bending losses occur whenever an optical fiber undergoes a bend of finite radius of curvature;

FIG. 17 shows a physical dimension of a sandwiched layer of the flexible optic fiber sensor film of the present invention;

FIG. 18 is a cross sectional view of the flexible optic fiber sensor film according to one embodiment of the present invention;

FIG. 19 is a cross sectional view of the flexible optic fiber sensor film according to one embodiment of the present invention;

FIG. 20 is a cross sectional view of the flexible optic fiber sensor film according to one embodiment of the present invention;

FIG. 21 shows a light loss signal detected by the light sensor in time domain, which has a sudden DC signal spike of the light loss signal;

FIG. 22 is an enlarged view of AC components of the light loss signal in time domain of FIG. 21;

FIG. 23 shows the AC components of the light loss signal of FIG. 22 in frequency domain;

FIG. 24 shows a light loss signal detected by the light sensor in time domain after the periodic AC components of the light loss signal, which has a sudden drop of the light loss signal;

FIG. 25 is a block diagram of a mat structure according to one embodiment of the present invention;

FIG. 26 is another block diagram of the mat structure according to one embodiment of the present invention.

### DETAILED DESCRIPTION

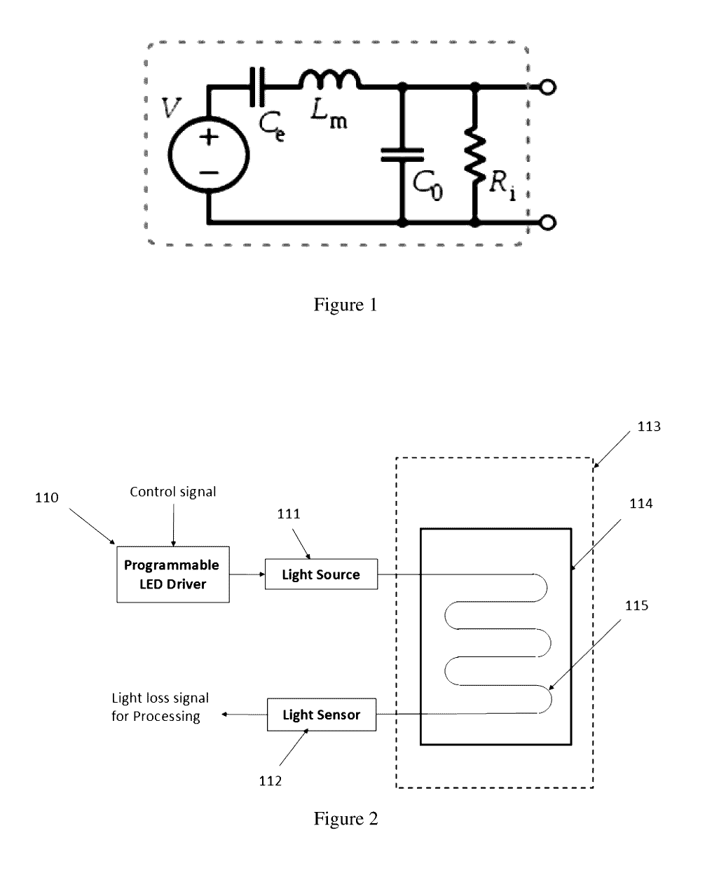

The objective of the present invention is to provide a flexible optic fiber sensor film 113 for measurement of respiration rate, heart rate, movement and presence of human subjects. As shown in FIG. 2, the flexible optic fiber sensor film 113 comprises a sandwiched layer 114 and an optic fiber cable 115. The optic fiber cable 115 is arranged in the sandwiched layer 114. A mat structure includes the flexible optic fiber sensor film 113, a programmable LED driver 110, a light source 111 and a light sensor 112. An output terminal of the programmable LED driver 110 is connected to the light source 111, and the light source 111 is connected to one terminal of the optic fiber cable 115, and the other terminal of the optic fiber cable 115 is connected to the light sensor 112. The programmable LED driver 110 is driven by a control signal to supply a LED current to the light source 111. The light source 111 is configured for generating light by the flow of the LED current and piping light into the optic fiber cable 115. The light sensor 112 is configured for detecting a light loss signal caused in the optic fiber cable 115. The light loss signal can be processed for the detection of presence, movement, respiration rate and heart rate of human subjects.

The flexible optic fiber sensor film 113 has the following characteristics:

1. The flexible optic fiber sensor film 113 is physically customizable in size to fit different applications. A sandwiched layer 114 can change in size depending on the type of application, and an optic fiber cable 115 can be routed in the sandwich layer 114 accordingly.

2. The sandwiched layer 114 is made of soft and flexible materials that can be embedded into a mattress or a pillow for a comfort feel and adaptable to the shape of a human body.

3. A sensor sensitivity of the flexible optic fiber sensor film 113 can be adjusted by changing a design of the sandwiched layer 114, and/or a specification of the optic fiber cable 115.

4. A programmable LED driver 110 is driven to supply LED current to a light source 111 to be configured for different weight loads of the flexible optic fiber sensor film 113. Based on the light loss signal, the LED driver may supply an appropriate current to the light source in order to compensate for light loss due to the heavier weight load. A higher LED current will increase light intensity piped into the optic fiber cable 115, enhancing the ability of the flexible optic fiber sensor film 113 to bear heavier loads.

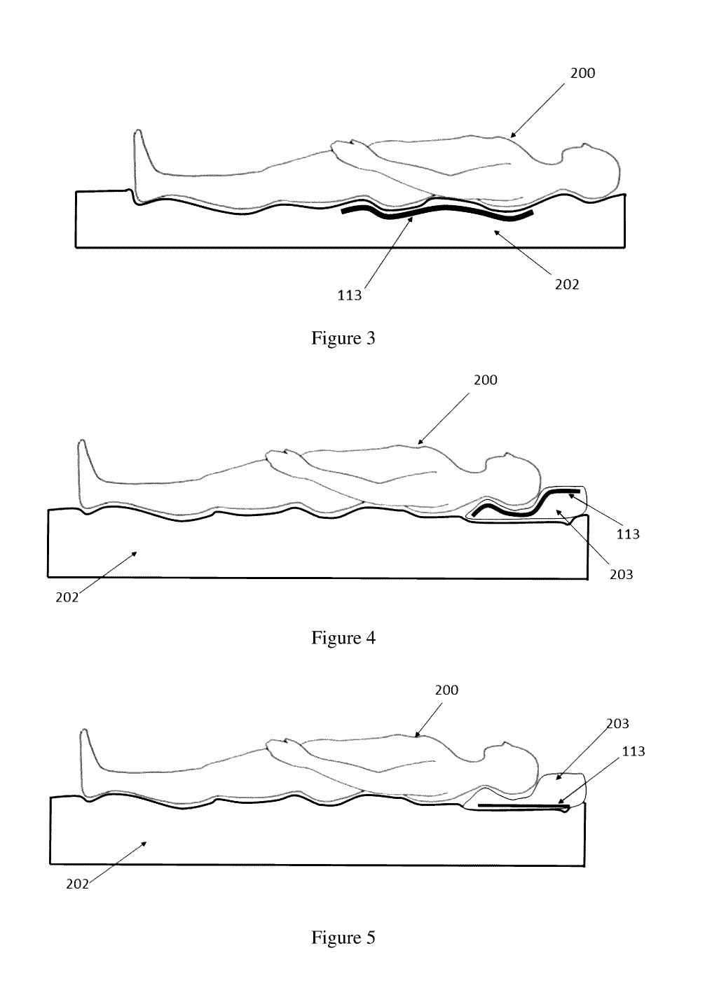

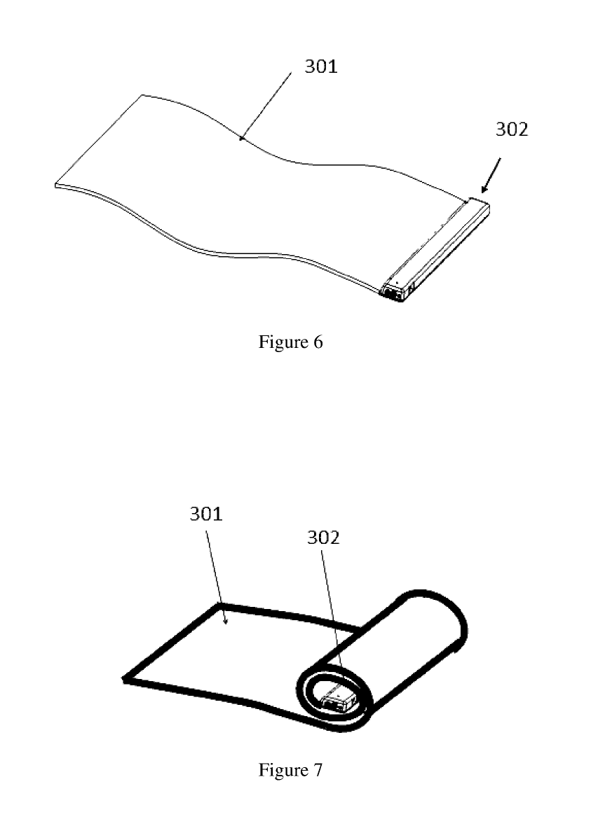

By utilizing polyethylene film as the sandwiched layer 114, the flexible optical fiber sensor film 113 is soft, flexible and comfortable enough to conform to the human body shape when the flexible optical fiber sensor film 113 is embedded into a mattress as shown in FIG. 3, embedded into and on the top of a pillow as shown in FIG. 4, embedded into and on the bottom of the pillow as shown in FIG. 5. Alternatively, the flexible optical fiber sensor film 113 may be a component of a flexible optic fiber sensor mat 301 of a mat structure which can be placed below the pillow or on top of the mattress. The mat structure is shown in FIGS. 6˜11. The sandwiched layer 114 can be configured with different orientations that will result in a different trade-off for a sensitivity and reliability for the flexible optic fiber sensor film 113.

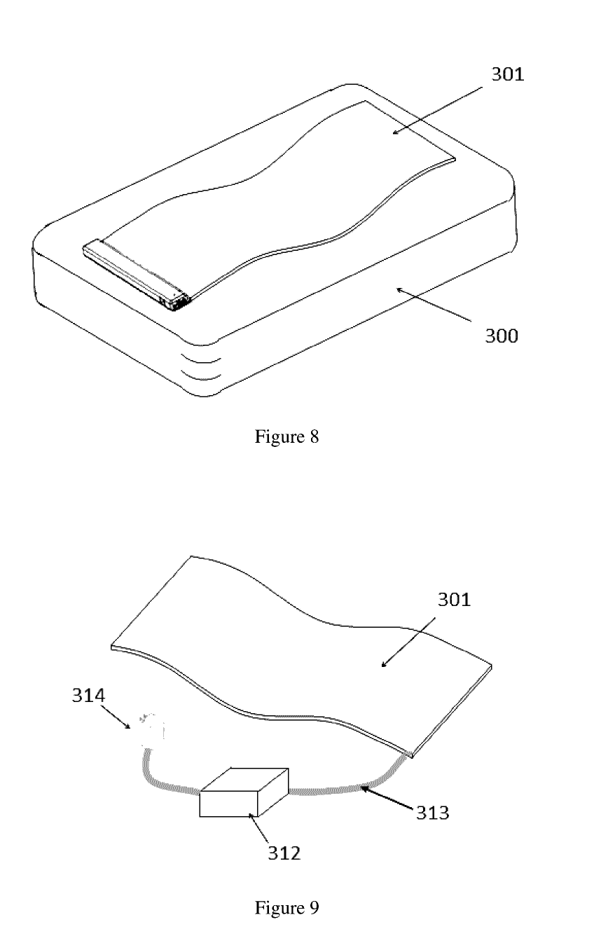

The flexible optic fiber sensor mat 301 can be applied in different applications for infant and adult monitoring. For infant monitoring, the flexible optic fiber sensor mat 301 can be attached to a head unit electronic assembly 302 that can act as a guide to roll up the flexible optic fiber sensor mat 301 to reduce space required for storage or shipment as shown in FIGS. 6 and 7. The head unit electronic assembly 302 also accommodates a dry cell battery as there should not be any AC/DC adapter attached to the flexible optic fiber sensor mat 301 for infant safety requirements. As shown in FIG. 8, the flexible optic fiber sensor mat 301 for infant monitoring is to be placed on top of an infant mattress 300 whereby the infant will sleep on top of the flexible optic fiber sensor mat 301 for monitoring.

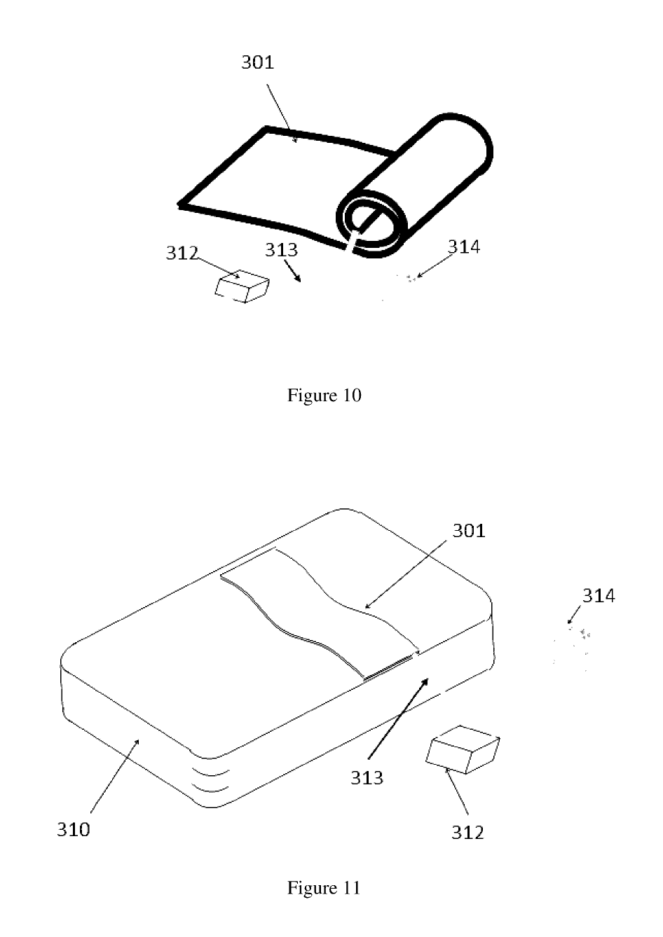

For adult monitoring, the mat structure includes an electronic box 312. And the flexible optic fiber sensor mat 301 is attached to the electronic box 312 via an optical fiber protective sleeve 313 as shown in FIGS. 9 and 10. The flexible optic fiber sensor mat 301 is placed across an adult mattress 310 as shown in FIG. 11. The electronic box 312 is powered via an AC/DC adapter 314 connected to a wall AC supply.

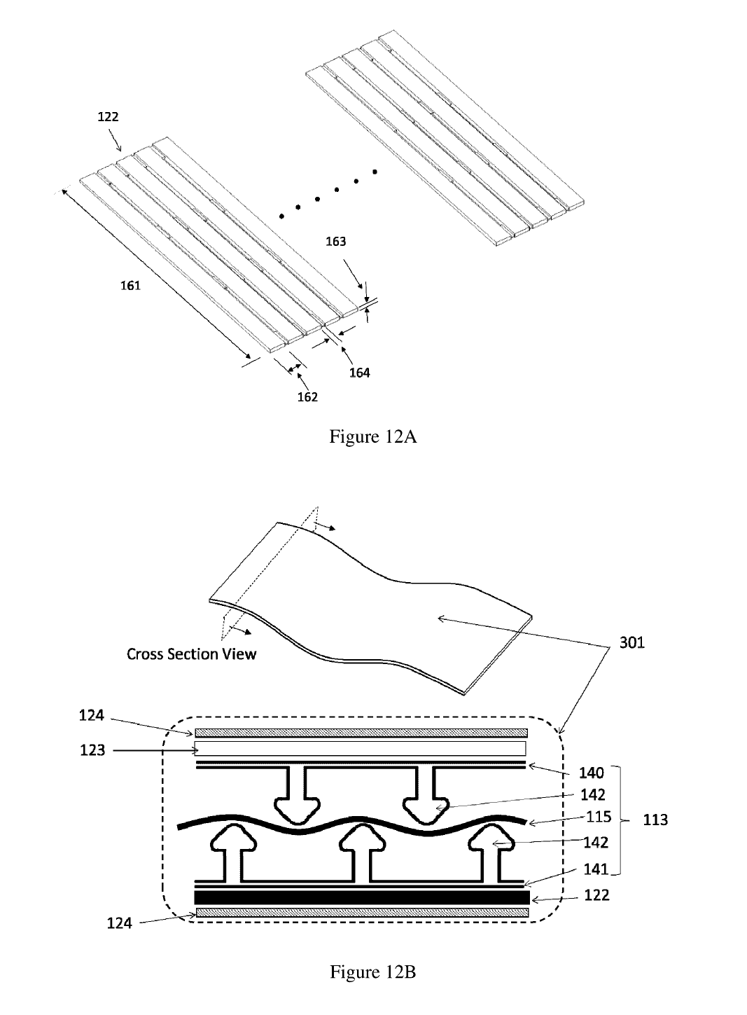

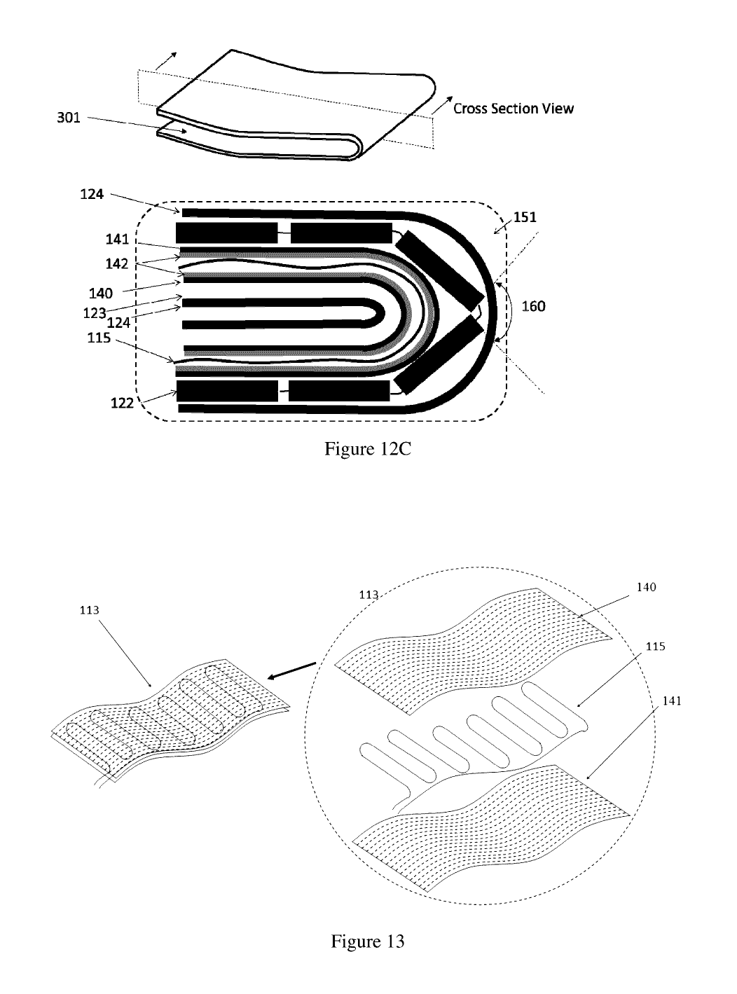

For the above infant and adult monitoring applications, the optical fiber cable 115 inside the flexible optic fiber sensor film 113 needs to be protected from breaking due to bending. To achieve this, as shown in FIGS. 12A˜12C, the flexible optic fiber sensor mat 301 may include a protective layer 122 below the flexible optic fiber sensor film 113. This protective layer 122 is designed to restrict a bending angle of the optic fiber cable 115 to be within its tolerated specification to prevent breakage. As shown in FIG. 12A, the protective layer has multiple strips with width 161 and length 162 spaced with gap 164 joined together and extends along the whole length and width of the flexible optic fiber sensor film 113. The gap 164 and thickness 163 controls the bending angle 160 of the optic fiber cable 115 within its tolerated limit when the flexible optic fiber sensor mat 301 is folded or bent. Another function of the protective layer 122 is to facilitate a rolling direction of the flexible optic fiber sensor mat 301. In the case whereby the flexible optic fiber sensor film 113 is embedded inside a mattress, this protective layer 122 is not necessary as the mattress cannot be bent. FIGS. 12B and 12C show two cross sectional views of the flexible optic fiber sensor mat 301. The flexible optic fiber sensor mat 301 may include a foam layer 123 on top of the flexible optic fiber sensor film 113. The foam layer 123 may provide more comfort when a human body is lying on top of it. The flexible optic fiber sensor mat 301 may further include an outer mat cover 124 that is waterproof to encase the flexible optic fiber sensor film 113, the foam layer 123 and the protective layer 122. The present invention discloses a flexible optic fiber sensor film 113 that can be embedded in a mattress, a pillow, or can be served as a component of a flexible optic fiber sensor mat 301 which can be placed on top of the mattress or below the pillow for sensing of respiration rate, heart rate, movement, presence of a human body lying on top of it.

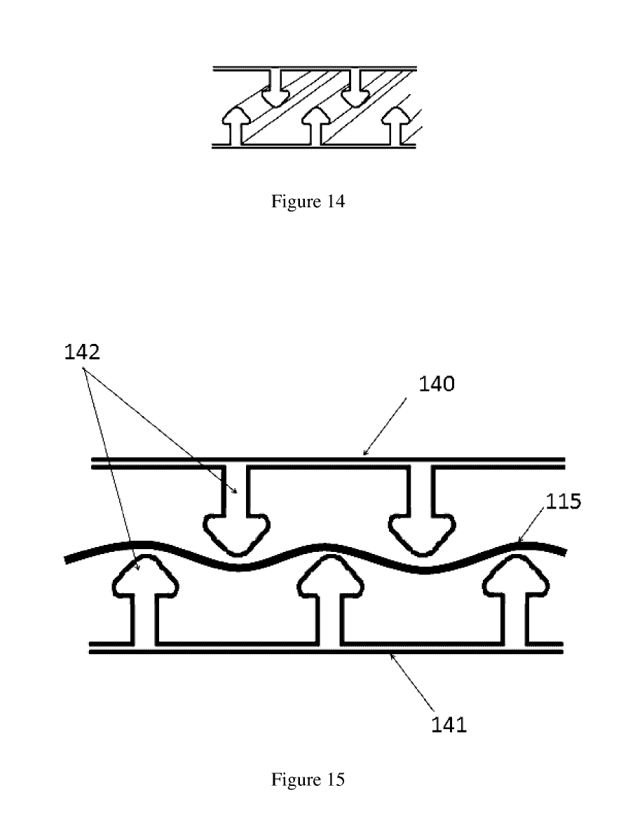

As shown in FIGS. 12B, 12C and 13, the sandwiched layer 114 includes an upper film 140 and a lower film 141. The upper and lower film 140 and 141 may be made of plastic, rubber, nylon or any other elastic material, particularly polyethylene. The optic fiber cable 115 is routed between the upper film 140 and the lower film 141. FIG. 13 shows an exploded view of the flexible optic fiber sensor film 113. FIG. 14 shows a perspective view of the sandwiched layer 114.

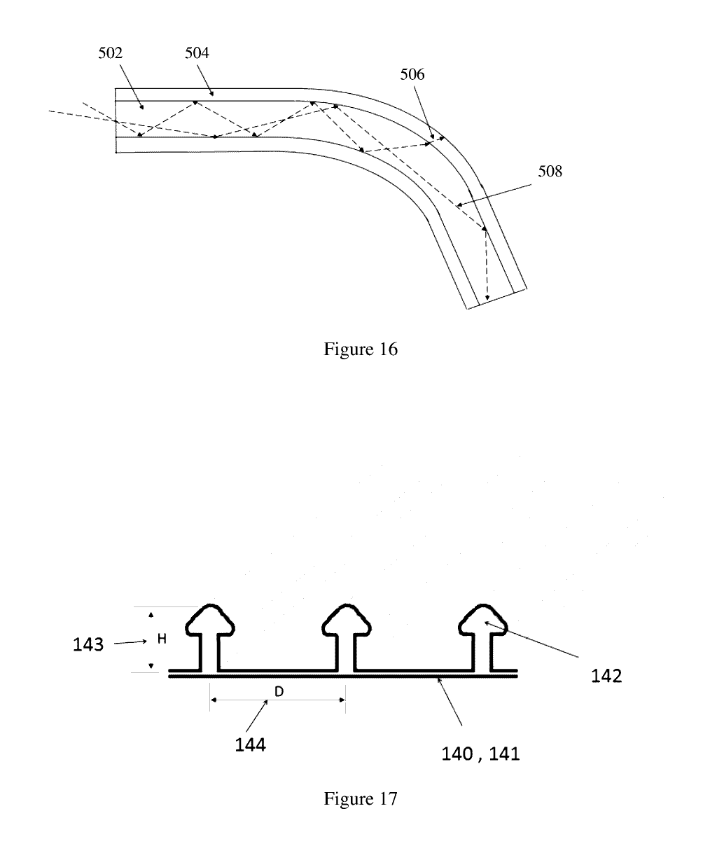

As shown in FIGS. 12B and 15, the flexible optic fiber sensor film 113 further includes protrusions 142 arranged on the upper and lower film 140 and 141 to abut against the optic fiber cable 115. The protrusions 142 will press and stress the optic fiber cable 115 and cause light loss in the optic fiber cable 115 when there are body movements of a human lying on top of the flexible optic fiber sensor film 113. The protrusions 142 are made of the same material of the upper and lower film 140 and 141, such as plastic, rubber, nylon or any other elastic material, particularly polyethylene. As shown in FIGS. 14 and 17, the protrusions 142 are multiple linear strips and its cross section is arrow-shaped. Alternatively, its cross section may be other shape, such as trapezoid, semi-circle, rectangular, etc. FIG. 16 shows bending losses occur whenever an optical fiber undergoes a bend of finite radius of curvature. If an external force is applied to either or both of the upper film 140 and the lower film 141, the optic fiber cable 115 is pressed by protrusions 142 and bent to cause light rays outside of the critical angle to be refracted out of the fiber core of the optic fiber cable 115, light losses occur. The flexible optic fiber sensor film 113 is tuned to be able to be configured for detecting a lung inhaling and exhaling cycle as well as a heartbeat of the human body 200 lying on top.

The sensitivity of the flexible optic fiber sensor film 113 is controlled by three parameters, namely the specification of the sandwiched layer 114, the configuration of the upper film 140 and the lower film 141, and the construction and the specification of the optic fiber cable 115. For the sandwiched layer 114, FIG. 17 shows the two parameters affecting the sensitivity of the flexible optic fiber sensor film 113, namely a height (H) 143 of the protrusions 142 and a distance (D) 144 between the adjacent protrusions 142. By varying these two parameters, the sensitivity of the flexible optic fiber sensor film 113 can be adjusted to fit applications that require different sensitivity levels. The experiment shows that the H/D ratio of 2/5 will achieve the best sensitivity and robustness. If H/D <2/5, the sensitivity will decrease. That means shorter protrusion height and wider distance between protrusions strips will cause sensitivity to decrease for the same optic fiber cable used. If H/D >2/5, the sensitivity will be greater, but the robustness will be affected as the fiber will have more stress due to the bigger bending angle.

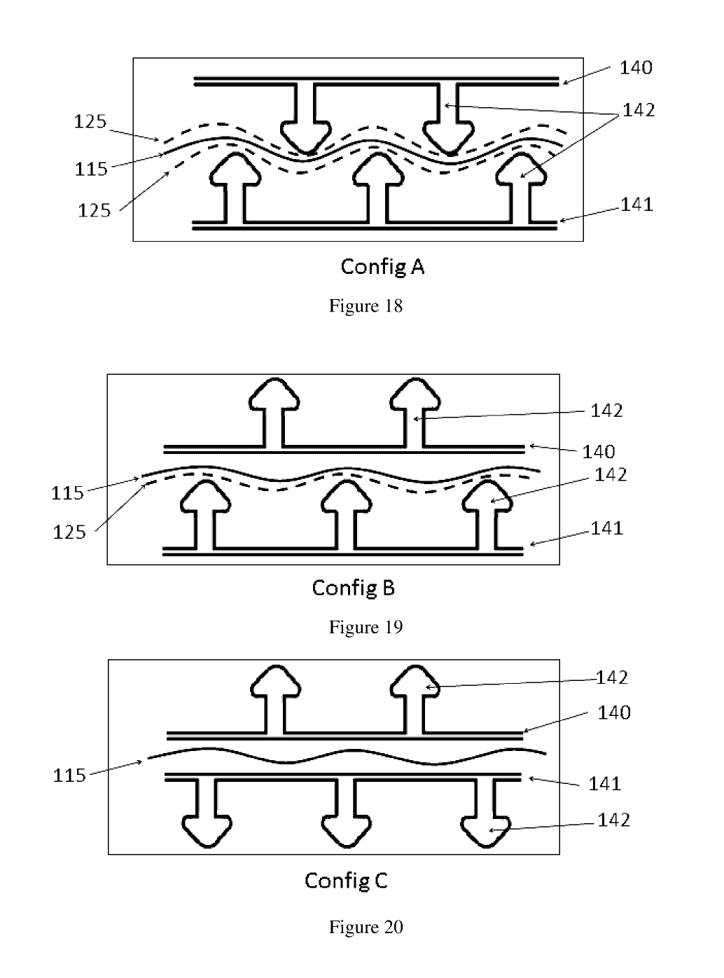

Additionally, FIGS. 18 to 20 show different configurations (Config A, Config B, Config C) of the flexible optic fiber sensor film 113.

As used to describe such embodiments, terms “face up”, “face down”, “face-to-face”, “back-to-back”, “upper” and “lower”, describe relative positions between the upper film 140 and the lower film 141. The term “face” as used herein refers to the protrusion 142, and the term “back” as used herein refers to the upper film 140 and the lower film 141. Further, it is understood that such terms do not necessarily refer to a direction defined by gravity or any other particular orientation. Instead, such terms are merely used to identify one portion versus another portion.

For Config A, as shown in FIG. 18, the protrusions 142 on the upper film 140 face down, and the protrusions 142 on the lower film 141 face up, so the protrusions on the upper and lower film 140 and 141 are face-to-face. All the protrusions 142 directly press onto the optic fiber cable 115. Config A produces the best sensitivity of the flexible optic fiber sensor film 113. However, Config A is the least reliable when an external sudden sharp force is applied to the flexible optic fiber sensor film 113. To make Config A less susceptible to breakage of the optic fiber cable 115, two pieces of protection films 125 are inserted in the sandwiched layer 114 to sandwich the optic fiber cable 115. The protection films 125 may be made of plastic, rubber, nylon or any other elastic material, particularly polyethylene. For Config B, both the protrusions 142 on the lower film 141 and the protrusions 142 on the upper film 140 face up. So only the protrusions 142 of the lower film 141 press directly onto the optical fiber cable 115. For Config B, the protrusions 142 on the upper film 140 do not come in contact with the optic fiber cable 115. In this case, only one piece of the protection film 125 is inserted in the sandwiched layer 114 and is between the optic fiber cable 115 and the lower film 141 to protect the optic fiber cable 115. For Config C, the protrusions 142 on the upper film 140 face up, and the protrusions 142 on the lower film 141 face down, so the upper film 140 and the lower film 141 are back-to-back and none of the protrusions 142 come into contact with the optic fiber cable 115. For Config C, no protection film 125 is needed to protect the optic fiber cable 115. A choice of Config A or B or C depends on the tradeoff of the sensitivity, the reliability of the flexible optic fiber sensor film 113 and cost of an additional protection film 125.

Another factor that affects the sensitivity of the flexible optic fiber sensor film 113 is the specification of the optic fiber cable 115. By selecting the optic fiber cable 115 with different refractive index, the sensitivity of the flexible optic fiber sensor film 113 can be adjusted.

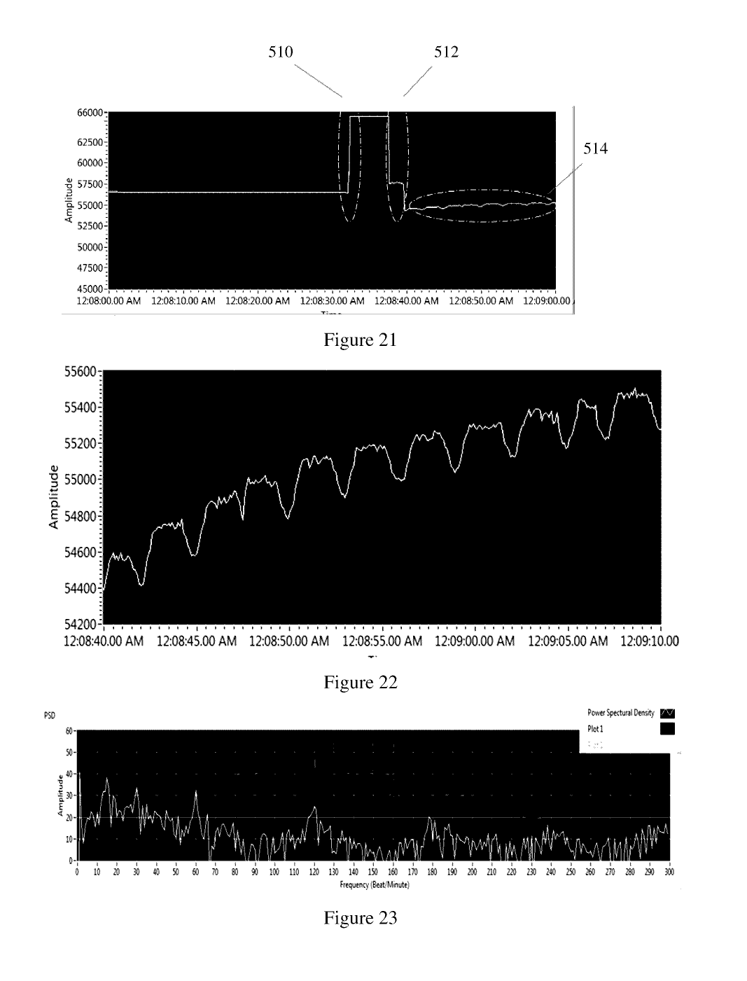

The flexible optic fiber sensor film 113 can be configured for detecting the presence of a human body 200 because the weight of the human body will cause light loss. FIG. 21 shows a light loss signal detected by the light sensor in time domain. Y-axis represents the light signal amplitude. The light loss will cause a sudden DC signal spike (DC means the signal bias level) of the light loss signal detected by a light sensor 112. As shown in FIG. 21, a DC signal spike is calibrated back by controlling the programmable LED driver 110 to pump current into the light source 111 to compensate for the light loss caused by the human body 200 lying on the flexible optic fiber sensor film 113. Subsequently, the human lung and heart fluctuations will generate AC components of the light loss signal received by the light sensor 112. AC component means the alternating signal sitting on the signal bias level (DC signal). These AC components of the light loss signal are the vital sign signal whereby respiration rate and heart rate data can be extracted.

FIG. 22 is an enlarged view of the AC components of the light loss signal in time domain of FIG. 21. Namely, FIG. 22 is an enlarged view of “Human Body Signal Detected” part in FIG. 21. These AC components of the light loss signal represent a collection of the human body's vital sign. In time domain, the AC components of the light loss signal can be clearly identified with each pulse represented as one breath count. FIG. 23 is an enlarged view of the AC components of the light loss signal of FIG. 21 in frequency domain. As shown in FIG. 23, to extract the heart rate signal, the AC components of the light loss signal are processed in frequency domain. By analyzing frequency harmonic peaks, we can deduce a heart rate signal in the frequency domain. As shown in FIG. 23, there are peaks at 60, 120, 180 and 240. We can deduce that the heart rate is 60 beats/minute and the peaks at 120, 180 and 240 are the 2nd, 3rd and 4th harmonics of the heart rate signal. The method for deriving heart rate signal is to look for the harmonic peaks to determine a sequence of 1st, 2nd, 3rd or 4th harmonics. We could stop at 3rd harmonics to deduce the heart rate if the 4th harmonics cannot be distinct.

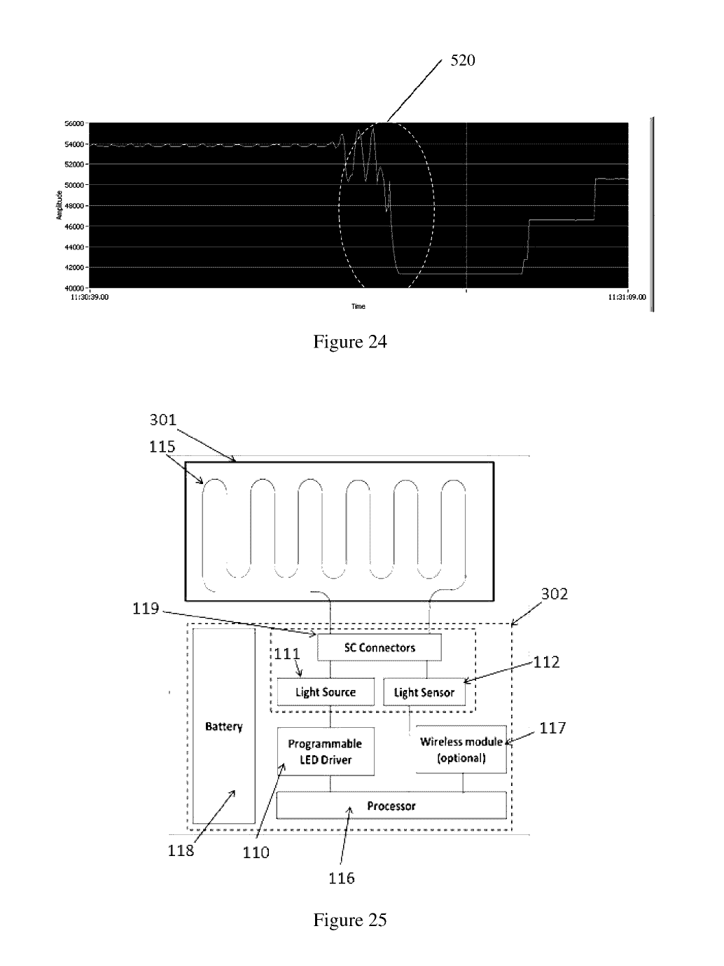

FIG. 24 shows a light loss signal detected by the light sensor in time domain after the time of FIG. 21. For the detection of presence of the human body, the light loss signal at the light sensor 112 is monitored for any sudden drop. As shown in FIG. 24, before the “Sudden drop of light loss signal”, the light sensor was detecting breath pattern. A sudden drop of the light loss signal after the periodic AC components of the light loss signal signifies that the human body 200 is not on top of the flexible optic fiber sensor mat 301 anymore. After the DC signal drop and after calibration, no breath pattern is detected; it means that human body is not present on top of the sensor.

FIG. 25 shows a block diagram of the mat structure. The mat structure further includes a head unit electronic assembly 302. The head unit electronic assembly 302 includes SC connectors 119, a light source 111, a light sensor 112, a programmable LED driver 110 and a processor 116. The flexible optic fiber sensor mat 301 is communicated to the head unit electronic assembly 302 by using the SC connectors 119. For infant applications, a dry cell battery 118 configured for supplying power to the programmable LED driver 110, the light sensor 112 and the processor 116 is built into the head unit electronic assembly 302 which is working together with the flexible optic fiber sensor mat 301 as one unit. Specifically, an input terminal of the programmable LED driver 110 is connected to the processor 116, and an output terminal of the programmable LED driver 110 is connected to the light source 111. The light source 111 is further connected to one terminal of the optic fiber cable 115 via the SC connectors 119, and the other terminal of the optic fiber cable 115 is connected to the light sensor 112 via the SC connectors 119; the light sensor 112 is connected to the processor 116. The processor 116 is configured for delivering a control signal to the programmable LED driver 110 to drive the programmable LED driver 110 to supply a LED current to the light source 111 and processing a light loss signal derived from the light sensor 112 for the detection of presence, movement, respiration rate and heart rate of human subjects. Advantageously, the head unit electronic assembly 302 further includes a wireless module 117. The wireless module 117 is optional and connected to the processor 116, which functions as a link to remote display devices such as smartphones and tablets etc. to process and display a signal status from the flexible optic fiber sensor mat 301.

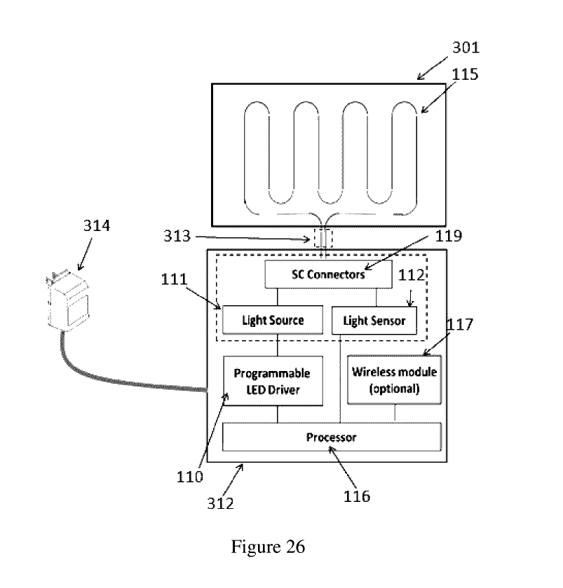

For adult applications, as shown in FIG. 26, an AC adapter 314 is used to supply power to the electronic box 312. The flexible optic fiber sensor mat 301 is connected to the electronic box 312 via a protective cable sleeve 313. The electronic box 312 includes a SC connectors 119, a light source 111, a light sensor 112, a programmable LED driver 110 and a processor 116. The flexible optic fiber sensor mat 301 is communicated to the electronic box 312 by using the SC connectors 119. Specifically, an input terminal of the programmable LED driver 110 is connected to the processor 116, and an output terminal of the programmable LED driver 110 is connected to the light source 111. The light source 111 is connected to one terminal of the optic fiber cable 115 via the SC connectors 119, and the other terminal of the optic fiber cable 115 is connected to the light sensor 112 via the SC connectors 119; the light sensor 112 is connected to the processor 116. The processor 116 is configured for delivering a control signal to the programmable LED driver 110 to drive the programmable LED driver 110 to supply a LED current to the light source 111 and processing a light loss signal derived from the light sensor 112 for the detection of presence, movement, respiration rate and heart rate of human subjects. Advantageously, the electronic box 312 further includes a wireless module 117. The wireless module 117 is optional and connected to the processor 116, which functions as a link to remote display devices such as smartphones and tablets etc. to process and display a signal status from the flexible optic fiber sensor mat 301.

In summary, this invention discloses a device for life sign measurement which consists of 5 major modules: a fiber sensing module, a detection module, an analysis module, a transmission module and a display module. The Fiber sensing module includes the flexible optic fiber sensor film 113. The detection module includes the programmable LED Driver 110, the light source 111 and the light sensor 112. The light sensor 112 is connected to an Analog to Digital Converter which converts the analog signal to digital form. This Analog to Digital Converter could reside as a standalone unit or be part of the processor 116 itself. The analysis module includes software algorithm executing in the processor 116 that analyses the digital signal from the Analog to Digital Converter in time domain and/or in frequency domain. After signal analysis, the result is provided to the transmission module (e.g. wireless module) for transmission to a display module. The display module could be a standalone device designed to display the result or a smartphone/tablet with Application running to display the result in a meaningful way. When implementing the present invention, the following advantageous effects can be achieved: the flexible optic fiber sensor film of the present invention can generate light loss for detection of presence, movement, respiration rate and heart rate of human subjects via strips of protrusions, and the present invention adopts the protection film to protect the optic fiber cable. The mat structure of the present invention adopts the head unit electronic assembly together with the flexible optic fiber sensor mat as one unit to be used for infant applications, and adopts the electronic box attached to the flexible optic fiber sensor mat via the optical fiber protective sleeve for adult applications. The invention can be configured for detection of presence, movement, respiration rate and heart rate of human subjects, and is safe and comfortable to the human subject.

While there has been illustrated and described what are presently considered to be preferred embodiments, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular embodiments disclosed, but that such claimed subject matter may also include all embodiments falling within the scope of the appended claims, and equivalents thereof.

1. A flexible optic fiber sensor film, comprising:
a sandwiched layer;
an optic fiber cable arranged in said sandwiched layer;
wherein said sandwiched layer comprises an upper film and a lower film; said optic fiber cable is sandwiched between said upper film and said lower film; protrusions are arranged on said upper film and said lower film to abut against said optic fiber cable and configured for generating light loss in said optic fiber cable when there are body movements of a human subject lying on top of said flexible optic fiber sensor film.

2. The flexible optic fiber sensor film according to claim 1, wherein said protrusions on said upper film and said protrusions on said lower film are face-to-face, to press directly onto said optical fiber cable.

3. The flexible optic fiber sensor film according to claim 2, wherein two pieces of protection films are inserted in said sandwiched layer and sandwich said optic fiber cable.

4. The flexible optic fiber sensor film according to claim 1, wherein both said protrusions on said upper film and said protrusions on said lower film face to one direction, such that only said protrusions on said upper film or only said protrusions on said lower film directly press onto said optic fiber cable.

5. The flexible optic fiber sensor film according to claim 4, wherein one piece of protection film is inserted in said sandwiched layer and is between said optic fiber cable and one of said upper film and said lower film such that said optic fiber cable does not contact said protrusions.

6. The flexible optic fiber sensor film according to claim 1, wherein said upper film and said lower film are back-to-back, such that none of the protrusions comes into contact with said optic fiber cable.

7. The flexible optic fiber sensor film according to claim 1, wherein said upper film, said lower film and said protrusions are made of elastic material selected from the group consisting of plastic, rubber, and nylon.

8. The flexible optic fiber sensor film according to claim 7, wherein said upper film, said lower film and said protrusions are made of polyethylene.

9. The flexible optic fiber sensor film according to claim 1, wherein a ratio H/D of the height H of said protrusion and the distance D between said protrusions is about 2/5.

10. The flexible optic fiber sensor film according to claim 1, wherein the shape of the cross section of said protrusion is selected from the group consisting of trapezoid, semi-circle, rectangular, and arrow-shaped.

11. The flexible optic fiber sensor film according to claim 2, wherein the ratio H/D of the height H of said protrusion and the distance D between said protrusions is about 2/5.

12. The flexible optic fiber sensor film according to claim 2, wherein the shape of the cross section of said protrusion is selected from the group consisting of trapezoid, semi-circle, rectangular, and arrow-shaped.

13. A mat structure, comprising:
a flexible optic fiber sensor film that comprises:
a sandwiched layer;
an optic fiber cable arranged in said sandwiched layer;
wherein said sandwiched layer comprises an upper film and a lower film; said optic fiber cable is sandwiched between said upper film and said lower film; protrusions are arranged on said upper film and said lower film to abut against said optic fiber cable and configured for generating light loss in said optic fiber cable when there are body movements of a human subject lying on top of said flexible optic fiber sensor film;
a processor;
a programmable LED driver connected between said processor and a light source;
a light source connected between an output terminal of said programmable LED driver and one terminal of said optic fiber cable;
a light sensor connected between the other terminal of said optic fiber cable and said processor;
wherein said processor is configured for delivering a control signal to drive said programmable LED driver to supply a LED current to said light source, said light source is configured for generating light by flow of said LED current and piping said light into said optic fiber cable; said light sensor is configured for detecting a light loss signal caused in said optic fiber cable, wherein said processor is also configured for processing said light loss signal derived from the light sensor for detection of vital signs.

14. The mat structure according to claim 13, wherein said processor, said programmable LED driver, said light source, and said light sensor are integrated into a head unit electronic assembly; said head unit electronic assembly further accommodates a dry cell battery configured for supplying power to said programmable LED driver, said light sensor and said processor.

15. The mat structure according to claim 13, wherein said processor, said programmable LED driver, said light source, and said light sensor are integrated into an electronic box; said flexible optic fiber sensor film is attached to the electronic box via an optical fiber protective sleeve; said electronic box is powered via an AC adapter connected to a wall AC supply.

16. The mat structure according to claim 14, further comprising:
a protective layer below said flexible optic fiber sensor film;
an outer mat cover, encasing said flexible optic fiber sensor film and said protective layer.

17. The mat structure according to claim 16, wherein said protective layer comprises multiple strips spaced with defined gaps in between.

18. The mat structure according to claim 16, further comprising a wireless module connected to said processor.

19-21. (canceled)
